(19) United States
(12) Patent Application Publication
(10) Pub. No.: US 2010/0040544 A1
(43) Pub. Date: Feb. 18, 2010

(54) METHOD FOR MANAGING PATIENTS WITH CANCER

(76) Inventors: Anne Waaler, Oslo (NO); Insa Flechsler, Oslo (NO)

Correspondence Address: GE HEALTHCARE, INC., IP DEPARTMENT 101 CARNEGIE CENTER, PRINCETON, NJ, 08540-6231, US

(21) Appl. No.: 12/514,317

(22) PCT Filed: Nov. 20, 2007
(86) PCT No.: PCT/NO07/00410

§ 371 Date: May 11, 2009

(30) Foreign Application Priority Data
Nov. 21, 2006 (NO) 20065342
Nov. 24, 2006 (NO) 20065406
Apr. 12, 2007 (NO) 20071894

(51) Int. Cl. A61K 51/00; A61B 5/055

(52) U.S. Cl. 424/1.81; 600/411

(57) ABSTRACT

The invention relates to methods for managing patients with cancers, more specifically to the planning of tumor treatment and monitoring treatment response, said management comprises MR imaging and/or MR spectroscopy with an MR imaging medium which comprises hyperpolarized ¹³C-pyruvate.

The invention relates to methods for managing patients with cancers, more specifically to the planning of tumor treatment and monitoring treatment response, said management comprises MR imaging and/or MR spectroscopy with an MR imaging medium which comprises hyperpolarized ¹³C-pyruvate.

Cancer is a class of diseases or disorders characterized by uncontrolled division of cells and the ability of these cells to invade other tissues, either by direct growth into adjacent tissue through invasion or by implantation into distant sites by metastasis. Cancer can affect people at all ages and it is one of the principal causes of death in developed countries.

Prostate cancer is the most common type of cancer in men, excluding skin cancer, and is the second leading cause of cancer death. Risk factors include age; incidence increases in men over the age of 65 years. It is more prevalent in the western world and 10% of the cases can be linked to family history. Prostate cancer is a continuum, progressing through localized, locally advanced, advanced and hormone-refractory stages. In general it is a slow growing cancer which is primarily under hormonal control, i.e. testosterone. Choice of treatment is dependent on the sate of disease and if detected early and treated appropriately, survival rates are excellent.

The presenting signs and symptoms of a prostate cancer vary as a function of its site of origin in the gland, and its extent of involvement. The majority of cancers arises in the peripheral zone and do not produce symptoms in the early stage of development. Those that arise in the transitional zone or that enlarge and encroach on the urethra, may produce hesitancy, a decrease in the force of urinary stream, intermittency and post-void leakage. However, all these symptoms may occur for other reasons, and there are no diagnostic symptoms or voiding pattern that can positively characterize prostate cancer. Due to improved public awareness and screening techniques, prostate cancer is being diagnosed in the earlier stages of development, often in men who are free of symptoms or asymptomatic.

A currently used screening procedure is PSA (prostate specific antigen) measurement. PSA is an enzyme secreted by the prostate gland and it is used as a marker for prostate disease. If not used for screening, PSA will usually be determined as part of the process of identifying someone likely to have prostate cancer. Especially tumors which are too small to be palpable on digital rectal examination (DRE) may only be suspected on PSA testing.

To confirm any findings of DRE/PSA, transrectal ultrasonography (TRUS) and TRUS guided needle biopsy are commonly used. However, especially for small localized tumors in the prostate the findings may be false-negative or false-positive: only 20-25% of cases of hypoechogenic images obtained with TRUS are confirmed to be prostate cancer. On the other hand, 25% of prostate tumors are isoechogenic. In biopsy usually 5 to 8 evenly spaced samples from different areas of the prostate are taken. In case of a small tumor, the biopsy needle may simply miss the tumor.

In WO-A-2006/011810 an in vivo MR imaging method was disclosed which allows for the discrimination between healthy and tumor tissue. An imaging medium comprising hyperpolarized ¹³C-pyruvate is used in the method.

Pyruvate is an endogenous compound which is very well tolerated by the human body, even in high concentrations. As a precursor in the citric acid cycle, pyruvate plays an important metabolic role in the human body. Pyruvate is converted into different compounds: its transamination results in alanine, via oxidative decarboxylation, pyruvate is converted into acetyl-CoA and bicarbonate, the reduction of pyruvate results in lactate and its carboxylation in oxaloacetate.

The metabolic conversion of hyperpolarized ¹³C-pyruvate to its metabolites hyperpolarized ¹³C-lactate, hyperpolarized ¹³C-bicarbonate (in the case of ¹³C₁-pyruvate, ¹³C_{1,2}-pyruvate or ¹³C_{1,2,3}-pyruvate only) and hyperpolarized ¹³C-alanine can be used for in vivo MR studying of metabolic processes in the human body. ¹³C₁-pyruvate has a T₁ relaxation in human full blood at 37° C. of about 42 s, however, the conversion of hyperpolarized ¹³C-pyruvate to hyperpolarized ¹³C-lactate, hyperpolarized ¹³C-bicarbonate and hyperpolarized ¹³C-alanine has been found to be fast enough to allow signal detection from the ¹³C-pyruvate parent compound and its metabolites. The amount of alanine, bicarbonate and lactate is dependent on the metabolic status of the tissue under investigation. The MR signal intensity of hyperpolarized ¹³C-lactate, hyperpolarized ¹³C-bicarbonate and hyperpolarized ¹³C-alanine is related to the amount of these compounds and the degree of polarization left at the time of detection, hence by monitoring the conversion of hyperpolarized ¹³C-pyruvate to hyperpolarized ¹³C-lactate, hyperpolarized ¹³C-bicarbonate and hyperpolarized ¹³C-alanine it is possible to study metabolic processes in vivo in the human or non-human animal body by using non-invasive MR imaging or MR spectroscopy.

The MR signal amplitudes arising from the different ¹³C-pyruvate metabolites vary depending on the tissue type. The unique metabolic peak pattern formed by alanine, lactate, bicarbonate and pyruvate can be used as fingerprint for the metabolic state of the tissue under examination and thus allows for the discrimination between healthy tissue and tumor tissue. Tumor tissue in ¹³C-MR images acquired of hyperpolarized ¹³C-pyruvate and its metabolites is indicated by the highest lactate signal or high weighted lactate over pyruvate or lactate over alanine signal, as described in detail in WO-A-2006/011810.

It has now been found that in vivo MR tumor imaging using an imaging medium that comprises hyperpolarized ¹³C-pyruvate may not only be used to discriminate between healthy and tumor tissue, but also for planning tumor treatment and monitoring the response of said treatment.

Thus the present invention provides a method of management of patients with cancer having a tumor, said method comprises
a) performing on the patient ¹³C-MR detection of the tumor and optionally of surrounding organs and/or surrounding area using an imaging medium that comprises hyperpolarized ¹³C-pyruvate, wherein signals of ¹³C-pyruvate and its ¹³C-containing metabolites alanine, lactate and optionally bicarbonate are detected; and
b) using the information obtained in step a) optionally together with other information obtained in investigations carried out to confirm diagnosis of cancer to determine the stage of disease.

In a preferred embodiment, the invention provides a method of management of patients with prostate cancer, said method comprises
a) performing on the patient ¹³C-MR detection of the prostate and optionally of surrounding organs and/or surrounding area using an imaging medium that comprises hyperpolarized ¹³C-pyruvate, wherein signals of ¹³C-pyruvate and its ¹³C-containing metabolites alanine, lactate and optionally bicarbonate are detected; and
b) using the information obtained in step a) optionally together with other information obtained in investigations carried out to confirm diagnosis of prostate cancer to determine the stage of disease.

The term “¹³C-MR detection” denotes ¹³C-MR imaging or ¹³C-MR spectroscopy or combined ¹³C-MR imaging and ¹³C-MR spectroscopy, i.e. ¹³C-MR spectroscopic imaging. The term further denotes ¹³C-MR spectroscopic imaging at various time points.

The terms “hyperpolarized” and “polarized” are used interchangeably hereinafter and denote a nuclear polarization level in excess of 0.1%, more preferred in excess of 1% and most preferred in excess of 0.1%, more preferably in excess of 1%, even more preferably in excess of 5% and most preferred in excess of 10%.

The level of polarization may for instance be determined by solid state ¹³C-NMR measurements in solid hyperpolarized ¹³C-pyruvate, e.g. solid hyperpolarized ¹³C-pyruvate obtained by dynamic nuclear polarization (DNP) of ¹³C-pyruvate or ¹³C-pyruvic acid (see WO2006/011809). The solid state ¹³C-NMR measurement preferably consists of a simple pulse-acquire NMR sequence using a low flip angle. The signal intensity of the hyperpolarized ¹³C-pyruvate in the NMR spectrum is compared with signal intensity of ¹³C-pyruvate in a NMR spectrum acquired before the polarization process. The level of polarization is then calculated from the ratio of the signal intensities of before and after polarization.

In a similar way, the level of polarization for dissolved hyperpolarized ¹³C-pyruvate may be determined by liquid state NMR measurements. Again the signal intensity of the dissolved hyperpolarized ¹³C-pyruvate is compared with the signal intensity of the dissolved ¹³C-pyruvate before polarization. The level of polarization is then calculated from the ratio of the signal intensities of ¹³C-pyruvate before and after polarization.

The term “signal” in the context of the invention refers to the MR signal amplitude or integral or peak area to noise of peaks in an MR spectrum which represent ¹³C-pyruvate and its ¹³C-containing metabolites alanine, lactate and optionally bicarbonate. In preferred embodiment, the signal is the peak area.

The term “signals of ¹³C-pyruvate and its ¹³C-containing metabolites alanine, lactate and optionally bicarbonate are detected” means that in the method of the invention the signals of ¹³C-pyruvate and ¹³C-alanine, or the signals of ¹³C-pyruvate and ¹³C-lactate, or the signals of ¹³C-pyruvate and ¹³C-alanine and ¹³C-lactate are detected, all of them optionally together with the signal of ¹³C-bicarbonate.

The term “imaging medium” denotes a liquid composition comprising hyperpolarized ¹³C-pyruvate as the MR active agent, i.e. imaging agent.

The term “¹³C-pyruvate” denotes a salt of ¹³C-pyruvic acid that is isotopically enriched with ¹³C, i.e. in which the amount of ¹³C isotope is greater than its natural abundance.

The isotopic enrichment of the hyperpolarized ¹³C-pyruvate used in the method of the invention is preferably at least 75%, more preferably at least 80% and especially preferably at least 90%, an isotopic enrichment of over 90% being most preferred. Ideally, the enrichment is 100%. ¹³C-pyruvate used in the method of the invention has to be isotopically enriched at least at the C1-position (in the following denoted ¹³C-pyruvate), since it is the C1-atom of pyruvate which is part of the carbon dioxide (and thus bicarbonate) generated by the PDH-catalyzed oxidation of pyruvate. Further, ¹³C-pyruvate used in the method of the invention may be isotopically enriched at the C1- and the C2-position (in the following denoted ¹³C_{1,2}-pyruvate), at the C1- and the C3-position (in the following denoted ¹³C_{1,3}-pyruvate) or at the C1-, C2- and C3-position (in the following denoted ¹³C_{1,2,3}-pyruvate). Isotopic enrichment at the C1-position only is preferred since ¹³C₁-pyruvate is readily available and has a favorably high T₁ relaxation in human full blood at 37° C. (about 42 s).

Methods for the preparation of imaging media comprising hyperpolarized ¹³C-pyruvate suitable in the methods of the invention are described in detail in WO-A-2006/011809, WO-A-2007/064226, WO-A-2007/069909 and WO-A-2007/111515; all of which are incorporated herein by reference.

Detailed descriptions on how to acquire ¹³C-MR images and/or ¹³C-MR-spectra with imaging media that comprise hyperpolarized ¹³C-pyruvate in order to distinguish between tumor tissue and healthy tissue can be found in WO-A-2006/011810 and in WO-A-2006/011809, all of which are incorporated herein by reference. For prostate imaging, a dedicated endorectal ¹³C-coil is preferably used.

The information obtained in step a) of the method of the invention may vary, depending on for instance the area which is covered by the ¹³C-MR detection procedure. If said procedure has been limited to the tumor itself, functional information of the tumor will be obtained but also anatomical information, i.e. tumor localization, size and delineation. Tumor tissue in the ¹³C-images and/or ¹³C-spectra obtained is indicated by a high lactate signal/peak. A method of in vivo MR tumor imaging using hyperpolarized ¹³C-pyruvate is described in detail in WO-A-2006/011810.

If the ¹³C-MR detection procedure has been extended to cover a greater area, i.e. surrounding organs and/or surrounding area of the tumor, information may be obtained as to whether the tumor has spread to regional lymph nodes or other neighboring tissues and organs. In the case of prostate cancer, if the ¹³C-MR detection procedure has been extended to cover a greater area i.e. including the pelvis or parts thereof, information may be obtained as to whether the tumor extends through the prostatic capsule, whether there are extra capsular extensions, whether regional lymph nodes are invaded and/or whether the tumor has invaded adjacent structures like for instance bladder neck or pelvic wall.

The information obtained in step a) is subsequently used in step b) to determine the stage of disease. Optionally, other information obtained in investigations carried out to confirm diagnosis of cancer may be used in step b). Investigations carried out to confirm diagnosis may include one or more of the following investigations: needle or surgical biopsy, histological grading, other imaging techniques like CT, PET or X-ray (e.g. mammography for breast cancer), testing of blood and body samples (e.g. PSA blood testing for prostate cancer, fecal occult blood testing for colorectal cancer and Pap smear for cervical cancer), cytogenetics and immunohistochemistry.

Investigations carried out to confirm diagnosis of prostate cancer may include one or more of the following investigations: PSA determination, digital rectal examination, transrectal ultrasonography (TRUS), (TRUS guided) needle biopsy and histological grading.

If the patient was diagnosed with (prostate) cancer by ¹³C-MR detection of the tumor and optionally of surrounding organs and/or the surrounding area using an imaging medium that comprises hyperpolarized ¹³C-pyruvate—either alone or optionally together with other investigations carried out to confirm diagnosis of (prostate) cancer, step a) of the method according to the invention needs not to be repeated. The information obtained by said ¹³C-MR detection of the prostate and optionally of surrounding organs and/or the surrounding area using an imaging medium that comprises hyperpolarized ¹³C-pyruvate would be sufficient to determine the stage of disease.

Cancer is usually a continuum, progressing through localized, locally advanced and advanced stages. Choice of treatment depends on the location and grade of the tumor, the general state of the patient and the stage of the disease. Generally, the stage of disease might be non-metastatic (localized or locally advanced) or metastatic. Prostate cancer is usually a slow growing cancer which is primarily under hormonal control, i.e. testosterone. Here, the stage of the disease might be non-metastatic (localized or locally advanced), metastatic or hormone refractory. Almost all prostate cancer tumors treated by hormonal therapy (androgen deprivation or androgen blockade) eventually achieve the ability to grow and progress in the absence of androgens. These are known as hormone-refractory (or hormone-independent or hormone-escaped) prostate cancer (HRPC). The prognosis for such disease is generally poor; however, there are a number of second-line treatments that may delay the worsening of symptoms.

In a preferred embodiment, the method of the invention comprises a step c), wherein a treatment is selected and the treatment is planned, said treatment being suitable for the stage of disease determined in step b).

Thus, in a second aspect the present invention provides a method of management of patients with cancer having a tumor, said method comprises
a) performing on the patient ¹³C-MR detection of the tumor and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using the information obtained in step a) optionally together with other information obtained in investigations carried out to confirm diagnosis of cancer to determine the stage of disease; and
c) selecting the treatment of the cancer.

In a preferred embodiment the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) use information obtained in step a) optionally together with other information obtained in investigations carried out to confirm diagnosis of prostate cancer to determine the stage of disease; and
c) selecting the treatment of the prostate cancer.

Generally, cancer treatment includes watchful waiting, surgery, radiation therapy, chemotherapy, immunotherapy, hormonal therapy, palliative care and investigational therapies. Watchful waiting is used for slow growing cancer types like prostate cancer and includes regular examination of the patient. Examples of surgical procedures for cancer include mastectomy for breast cancer and prostactomy for prostate cancer. Radiation therapy is the use of ionizing radiation to kill cancer cells and shrink tumors. It may be used to treat almost every kind of solid tumor, including tumors of the brain, breast, lung, pancreas and prostate. Chemotherapy is the treatment of cancer with drugs that can destroy cancer cells. Most forms of chemotherapy target all rapidly dividing cells and are not specific for cancer cells. In immunotherapy, immune mechanisms are used against tumors. The agents are monoclonal antibodies directed against proteins that are characteristic to the cells of the cancer in question, or cytokines that modulate the immune system's response. Immunotherapy may for instance be used in the treatment of breast cancer. The growth of some cancers, e.g. breast and prostate cancer, can be inhibited by providing or blocking certain hormones and hormonal therapy might be used in the treatment of such cancers. Palliative care deals with the control of the symptoms of cancer like nausea, pain, vomiting or hemorrhage and plays an important role in the quality of life of cancer patients. Investigational therapies use new treatments for cancer patients which are not yet approved and such treatments are carried out in clinical trials.

The term “treatment” although mainly being used in the singular form in this application may include the combination of different treatments, e.g. combinations like surgery and radiation or radiation and hormonal therapy.

If method is of the invention is related to management of prostate cancer patients and if the stage of the disease in step b) has been determined to be non-metastatic, the treatment is preferably selected from watchful waiting, radiotherapy with/without hormonal therapy, radical prostactomy with/without hormonal therapy, hormonal therapy, preferably for locally advanced prostate cancers or investigational approaches, preferably for localized prostate cancers.

If method is of the invention is related to management of prostate cancer patients and if the stage of the disease in step b) has been determined to be metastatic, the treatment is preferably selected from hormonal therapy, e.g. therapy with LHRHa (luteinising hormone-releasing hormone antagonists), anti-androgens (intermittent or total androgen blockade) or combined LHRHa/anti-androgen therapy or orchidectomy (surgical castration).

If method is of the invention is related to management of prostate cancer patients and if the stage of the disease in step b) has been determined to be hormone refractory, treatment is preferably selected from chemotherapy, palliative care or investigational therapies. Agents used in investigational therapies are for instance endothelin antagonists, immunotherapeutics or agents that prohibit or decrease tumor angiogenesis.

Hence, in a third aspect the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease; and
c) if the stage of the disease has been determined to be non-metastatic in step b), selecting a treatment of the prostate cancer from the group consisting of watchful waiting, radiotherapy with/without hormonal therapy, radical prostactomy with/without hormonal therapy and hormonal therapy.

In a forth aspect, the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease; and
c) if the stage of the disease has been determined to be metastatic in step b), selecting a treatment from the group consisting of hormonal therapy and orchidectomy.

In a fifth aspect, the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease; and
c) if the stage of the disease has been determined to be hormone refractory in step b) selecting a treatment from the group consisting of palliative care and investigational therapy.

Surgery is often used as a therapy if the stage of the disease is non-metastatic. The goal of surgery can either be the removal of only the tumor, the entire organ and regional lymph nodes. Often surgery leads to decrease of life quality (e.g. disfiguration as a consequence of mastectomy or colostomy after surgery of colorectal cancer. For non-metastatic prostate cancer, a common treatment is radical prostactomy. This operation involves removing the entire gland including some of the nearby tissues. Radical prostactomy can cause problems like incontinence and impotence. Thus, in the methods of the invention, if the stage of the disease has been determined to be non-metastatic and the tumor is localized, minimal-invasive or non-invasive procedures are preferred.

In a preferred embodiment, minimal-invasive or non-invasive procedures are selected from cryosurgery, laser treatment and high intensity focused ultrasound, with high intensity focused ultrasound being preferred.

Cryosurgery is carried out in form of cryosurgical ablation, wherein a cryogenic liquid, usually liquid nitrogen is either applied on the diseased tissue (tumor) or wherein a cryogenic liquid is circulated within a so-called cryogenic probe. In cryosurgery of prostate cancer, cryogenic probes are inserted into the prostate via the perineum and liquid nitrogen is circulated through them, producing local temperatures of about −18° C. destroying local tissue (intracellular necrosis begins at −2° C.).

In laser treatment, a laser is used that produces transient temperatures within and around tumor tissue of greater than 60° C., leading to rapid tissue destruction.

High intensity focused ultrasound (HIFU) uses the physical effect of ultrasound, which results in thermal tissue destruction, with high precision and minimal damage to surrounding structures. The ultrasound is focused in a manner similar to how a magnifying glass focuses light. The ultrasound waves are directed from a transducer into a small focal volume. During treatment, the beam of focused ultrasound energy penetrates through soft tissue and produces well defined regions of protein denaturation, irreversible cell damage, and coagulative necrosis, at specific target locations. A single exposure of focused ultrasound energy is called a “sonication.” Multiple sonications are necessary to ablate the targeted tissue. Tight focusing is designed to limit the ablation to the targeted location. This non-invasive method is preferably carried out under MR guidance.

Prior to the treatment, anatomical MR images, capable of showing the tumor and surrounding tissue and/or organs, are used to position the patient and plan the HIFU treatment, i.e. to determine the optimal path to the tumor for the focused ultrasound beam. In a preferred embodiment, the information from the ¹³C-MR detection in step a) of the method of the invention is used to position and plan the HIFU treatment.

As the treatment is performed, a MR thermal mapping system displays the relative tissue temperature as a color map superimposed on an anatomical MR image. This allows the physician to observe temperature changes inside the body in real time during treatment. Based on these observed temperature changes, the physician can adjust treatment parameters accordingly to ensure safe and effective thermal ablation. After each sonication, the transducer and MR scan plane are preferably automatically directed to the succeeding point, and the process is repeated until the entire target volume has been treated.

Immediately after the treatment, i.e. following the treatment, anatomical MR images can be used to evaluate treatment outcome. T₁ weighted MR imaging with gadolinium based contrast agents is often used to determine which regions have become ablated. Regions which do not have contrast agent uptake are considered “non-perfused” or destroyed by the thermal effects of the focused ultrasound.

In a preferred embodiment following the treatment, ¹³C-MR images and/or ¹³C-MR spectra of the tumor, preferably a tumor in the prostate, using an imaging medium that comprises hyperpolarized ¹³C-pyruvate are acquired to evaluate the HIFU treatment. Regions which do not show metabolism, i.e. regions which upon administration of an imaging medium that comprises hyperpolarized ¹³C-pyruvate only show the ¹³C-pyruvate signal and no signals from possible metabolites indicate an area with cells which are destroyed by the HIFU treatment. If prior to the treatment ¹³C-MR images and/or ¹³C-MR spectra have been acquired using an imaging medium that comprises hyperpolarized ¹³C-pyruvate, these images and/or spectra may be compared with images and/or spectra after the HIFU treatment. Effective HIFU treatment may then also be determined by the decrease or the absence of the ¹³C-lactate signal which, as explained above, is an indicator of tumor tissue and/or by the ¹³C-pyruvate signal and absent signals of metabolites.

By acquiring ¹³C-MR images and/or ¹³C-MR spectra using an imaging medium that comprises hyperpolarized ¹³C-pyruvate prior and after the HIFU treatment the physician is able to determine whether the treatment was successful or only partially successful. In the former case regions which do not show metabolism, i.e. regions which upon administration of an imaging medium that comprises hyperpolarized ¹³C-pyruvate only show the ¹³C-pyruvate signal and no signals from possible metabolites indicate an area with cells which are destroyed. A partially successful treatment would be indicated by a decrease in ¹³C-lactate signal, i.e. a decreased metabolism but metabolism would still be detectable. In the latter case, the HIFU treatment may be repeated.

In a sixth aspect, the present invention provides a method of management of patients with non-metastatic cancer having a tumor, said method comprises
a) acquiring MR images and/or MR spectra of the tumor and optionally of surrounding organs and/or the surrounding area;
b) performing thermal ablation treatment of the tumor using MR guided high intensity focused ultrasound; and
c) following the treatment, performing on the patient ¹³C-MR detection of the tumor and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to evaluate the treatment of step c).

In one embodiment of the method of the previous paragraph, anatomical MR images (proton images) are acquired in step a), either with or without a MR imaging medium, e.g. a paramagnetic metal chelate like for instance a gadolinium chelate like Omniscan™, (GE Healthcare). In another embodiment, ¹³C-MR detection is performed, i.e. ¹³C-MR images and/or ¹³C-MR spectra are acquired using an imaging medium that comprises hyperpolarized ¹³C-pyruvate wherein signals of ¹³C-pyruvate and its ¹³C-containing metabolites alanine, lactate and optionally bicarbonate are detected. By doing so, metabolic information of the area under examination is obtained. In a third embodiment, these two embodiments are combined, i.e. MR images (proton images) may be acquired using a paramagnetic metal chelate to get anatomical information and to determine the location of the tumor and ¹³C-MR images and/or ¹³C-MR spectra may be acquired using an imaging medium that comprises hyperpolarized ¹³C-pyruvate which will provide detailed information about the metabolic activity of the tissue and thus allow for discrimination between tumor tissue and healthy tissue.

In a further preferred embodiment, the method of the previous paragraph is a method of management of patients with non-metastatic prostate cancer.

If the evaluation in step c) reveals, that the tumor has not been fully destroyed, steps b) and c) may be repeated until the desired result is achieved.

Yet another aspect of the invention is an array of devices in a MR scanner room comprising a MR scanner, a patient table, a DNP polarizer, an injector and a transducer that generates focused ultrasound beams.

In a preferred embodiment, the invention provides an array of devices in a MR scanner room comprising a MR scanner, a patient table docked to the MR scanner, a DNP polarizer for producing hyperpolarized MR imaging agent, an injector connected to said DNP polarizer for injecting the hyperpolarized MR imaging agent into a patient on said patient table and a transducer that generates focused ultrasound beams.

In a further preferred embodiment, said transducer is incorporated into said patient table and said patient table further comprises an automated positioning system. In another preferred embodiment, said transducer is incorporated into the dedicated endorectal ¹³C-coil which is preferably used for imaging of the prostate according to the methods of the invention.

Suitable DNP polarizers are described in WO-A-02/37132. The process of producing hyperpolarized MR imaging agents using a DNP polarizer is disclosed in detail in WO-A-99/35508, WO-A-02/37132 and WO-A-2006/011809, all of which are included by reference therein.

Usually, tumor treatment response of any of the aforementioned treatments, i.e. also HIFU tumor treatment, may be monitored by obtaining sequential MR, CT or PET images at certain intervals after the treatment has been commenced. Treatment response is documented by decrease in tumor size and decrease in tumor enhancement. In many cases, a detectable change in tumor size is observed only after a significant long period of time, for example after treatment for a period of 3 month and more (e.g. chemotherapy or radiotherapy) or 3 month from the treatment (surgery, laser treatment or HIFU). Such long periods of time could be harmful to the patient for during this long period of time, tumor cells could multiply and/or metastasize, and lead to worsening the patient's condition.

Thus it is preferred to monitor tumor treatment by ¹³C-MR detection using an imaging medium that comprises hyperpolarized ¹³C-pyruvate. By doing so, early treatment response can be monitored which does not manifest itself in a detectable decrease in tumor size but which is detectable by slowing down metabolism in the tumor and thus a decrease in the ¹³C-lactate signal.

In a preferred embodiment, ¹³C-MR images and/or ¹³C-MR spectra of the tumor or preferably the prostate using an imaging medium that comprises hyperpolarized ¹³C-pyruvate are acquired early after treatment to monitor early treatment response and these images and/or spectra are compared with ¹³C-MR images and/or ¹³C-MR spectra that were acquired using an imaging medium that comprises hyperpolarized ¹³C-pyruvate images prior to the treatment. Treatment response is then determined by comparing the ¹³C-lactate signal of the acquired ¹³C-MR images and/or ¹³C-MR spectra prior and after said treatment. A decrease in the ¹³C-lactate signal indicates a slow-down in the metabolism of the tumor and thus a response to treatment. Moreover, cells or regions of cells which are destroyed by the treatment may be identified by a relatively high ¹³C-pyruvate signal and the absence of signals of ¹³C-metabolites.

In accordance with the present inventive methods, early treatment response can be measured within a period of about 168 hours, preferably about 72 hours, more preferably about 48 hours, even more preferably about 24 hours and most preferably about 12 hours, from the commencement of said treatment. For example the response can be monitored every 12, 24, 36, 48, 60, 72, 84, 96, 108, 120, 132, 144, 156 or 168 hours, or any combination thereof, from the commencement (hormonal treatment, radiation therapy or chemotherapy) or finishing (surgery, laser treatment or HIFU) of the treatment. For chemotherapy, response to chemotherapy is measured preferably within a period of about 72 hours, more preferably about 48 hours and even more preferably about 24 hours from the commencement of the chemotherapy. For hormonal treatment, response to hormonal treatment is measured preferably within a period of about 168 hours, more preferably 144 hours and even more preferably 120 hours from the commencement of the hormonal treatment.

Thus, in a seventh aspect the present invention provides a method of management of patients with cancer having a tumor, said method comprises
a) performing on the patient ¹³C-MR detection of the tumor and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of cancer to determine the stage of disease;
c) selecting the treatment of the cancer;
d) carrying out treatment; and
e) repeating step a) to monitor the early treatment response.

The method of the previous paragraph is preferably a method of management of patients with prostate cancer and said method thus comprises performing ¹³C-MR detection of the prostate in step a).

The monitoring of the early treatment response is done by comparing the results of the ¹³C-MR detection of step a) and step e), more preferably by comparing the ¹³C-lactate signal of the ¹³C-MR detection of step a) and step e). A decrease in the ¹³C-lactate signal indicates a slow-down in the metabolism of the tumor and thus a response to treatment. Moreover, cells or regions of cells which are destroyed by the treatment may be identified by a relatively high ¹³C-pyruvate signal and the absence of signals of ¹³C-metabolites.

If step e) reveals that the treatment has not been fully successful, steps d) and e) may be repeated (follow-up treatment) until the desired results are achieved. The follow-up treatment may or may not be identical with the initial treatment. As an example, initial treatment may be surgery, e.g. removal of the tumor and the follow-up treatment may be radiation therapy to destroy any remaining cancer cells.

Since monitoring of early treatment response is possible with the use of hyperpolarized ¹³C-pyruvate as MR imaging agent in a ¹³C-MR detection procedure, this compound may be used in clinical (phase 1) trials to give an early indication of drug efficacy of potential chemotherapeutic agents.

In another embodiment, the method according to the seventh aspect of the invention is a method of monitoring early treatment response of patients with prostate cancer, the method comprising ¹³C-MR detection of the prostate and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected.

A more preferred embodiment of the present invention according to the seventh aspect is a method of management of patients with non-metastatic cancer having a tumor, said method comprises
a) acquiring MR images and/or MR spectra of the tumor and optionally of surrounding organs and/or the surrounding area;
b) performing thermal ablation treatment of the tumor using MR guided high intensity focused ultrasound; and
c) following the treatment, performing on the patient ¹³C-MR detection of the tumor and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to evaluate the treatment of step b); and
d) repeating step c) to monitor the early treatment response.

In a eighth aspect the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease; and
c) if the stage of the disease has been determined to be non-metastatic in step b), selecting a treatment of the prostate cancer from the group consisting of watchful waiting, radiotherapy with/without hormonal therapy, radical prostactomy with/without hormonal therapy and hormonal therapy;
d) carrying out treatment; and
e) repeating step a) to monitor the early treatment response.

If step e) reveals that the treatment has not been fully successful, steps d) and e) may be repeated (follow-up treatment) until the desired results are achieved. The follow-up treatment may or may not be identical with the initial treatment.

A preferred embodiment of the method according to the eighth aspect the invention is a method of management of patients with non-metastatic prostate cancer, said method comprises
a) selecting a treatment of the prostate cancer from the group consisting of watchful waiting, radiotherapy with/without hormonal therapy, radical prostactomy with/without hormonal therapy and hormonal therapy;
b) carrying out treatment; and
c) performing on the patient ¹³C-MR detection of the prostate and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to monitor the early treatment response.

If step c) reveals that the treatment has not been fully successful, steps b) and c) may be repeated (follow-up treatment) until the desired results are achieved. The follow-up treatment may or may not be identical with the initial treatment.

In a preferred embodiment of the methods according to the eighth aspect of the invention, the selected treatment is minimal invasive or non-invasive, more preferably cryosurgery, laser treatment or high intensity focused ultrasound (HIFU), most preferably MR guided high intensity focused ultrasound.

In a ninth aspect, the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) use information obtained in step a) optionally together with other information obtained in investigations carried out to confirm diagnosis of prostate cancer to determine the stage of disease;
c) if the stage of the disease has been determined to be metastatic in step b), selecting treatment from the group consisting of hormonal therapy and orchidectomy;
d) carrying out treatment; and
e) repeating step a) to monitor the early treatment response.

If step e) reveals that the treatment has not been fully successful, steps d) and e) may be repeated (follow-up treatment) until the desired results are achieved. The follow-up treatment may or may not be identical with the initial treatment.

A preferred embodiment of the present invention according to the ninth aspect is a method of management of patients with metastatic prostate cancer, said method comprises
a) selecting a treatment from the group consisting of hormonal therapy and orchidectomy;
b) carrying out treatment; and
c) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected to monitor the early treatment response.

If step c) reveals that the treatment has not been fully successful, steps b) and c) may be repeated (follow-up treatment) until the desired results are achieved. The repetitive treatment may or may not be identical with the initial treatment.

In a tenth aspect, the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease;
c) if the stage of the disease has been determined to be hormone refractory in step b) selecting treatment from the group consisting of chemotherapy and investigational therapy;
d) carrying out treatment; and
e) repeating step a) to monitor the early treatment response.

If step e) reveals that the treatment has not been fully successful, steps d) and e) may be repeated (follow-up treatment) until the desired results are achieved. The follow-up treatment may or may not be identical with the initial treatment.

A preferred embodiment of the present invention according to the tenth aspect is a method of management of patients with hormone refractory prostate cancer, said method comprises
a) selecting a treatment from the group consisting of chemotherapy and investigational therapy;
b) carrying out treatment; and
c) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected to monitor the early treatment response.

If step c) reveals that the treatment has not been fully successful, steps b) and c) may be repeated (follow-up treatment) until the desired results are achieved. The follow-up treatment may or may not be identical with the initial treatment.

Performing ¹³C-MR detection on patients with cancer using a hyperpolarized MR imaging agent that comprises ¹³C-pyruvate is not only useful for monitoring early treatment response, but also for a general monitoring of treatment response, i.e. monitoring treatment response within a period of more than 168 hours after commencement or finishing (surgery, laser treatment or HIFU) of the treatment. In a preferred embodiment, said period is 3 to 12 month after commencement or finishing of the treatment.

Thus, in a eleventh aspect the present invention provides a method of management of patients with cancer having a tumor, said method comprises
a) performing on the patient ¹³C-MR detection of the tumor and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of cancer to determine the stage of disease;
c) selecting the treatment of the cancer;
d) carrying out treatment; and
e) repeating step a) to monitor the treatment response.

The method of the previous paragraph is preferably a method of management of patients with prostate cancer and said method thus comprises ¹³C-MR detection of the prostate in step a) and e).

If step e) reveals that the treatment has not been fully successful, steps d) and e) may be repeated (follow-up treatment) until the desired results are achieved. The follow-up treatment may or may not be identical with the initial treatment.

In another embodiment, the method according to the tenth aspect of the invention is a method of monitoring treatment response of patients with cancer having a tumor and receiving or having received treatment for said cancer, the method comprising performing on the patient ¹³C-MR detection of the tumor or the area the tumor had been located before treatment and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected. In a preferred embodiment, the method according to the tenth aspect of the invention is a method of monitoring treatment response of patients with prostate cancer and performing on the patient ¹³C-MR detection of the prostate and optionally of surrounding organs and/or the surrounding area.

Again treatment response manifests itself by decrease in tumor size and decrease in tumor enhancement which can be seen on the ¹³C-MR images. Further treatment response can be seen from a slow-down of metabolism in the tumor and thus a decrease in the ¹³C-lactate signal in the ¹³C-MR images and or ¹³C-MR spectra. Treatment response may also be determined by comparing the ¹³C-lactate signal prior and after said treatment. A decrease in the ¹³C-lactate signal indicates a slow-down in the metabolism of the tumor and thus a response to treatment. Moreover, cells or regions of cells which are destroyed by the treatment may be identified by a relatively high ¹³C-pyruvate signal and the absence of signals of ¹³C-metabolites.

In an twelfth aspect the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease; and
c) if the stage of the disease has been determined to be non-metastatic in step b), selecting a treatment of the prostate cancer from the group consisting of watchful waiting, radiotherapy with/without hormonal therapy, radical prostactomy with/without hormonal therapy and hormonal therapy;
d) carrying out treatment; and
e) repeating step a) to monitor treatment response.

If step e) reveals that the treatment has not been fully successful, steps d) and e) may be repeated (follow-up treatment) until the desired results are achieved. The follow-up treatment may or may not be identical with the initial treatment.

A preferred embodiment of the method according to the twelfth aspect the invention is a method of management of patients with non-metastatic prostate cancer, said method comprises
a) selecting a treatment of the prostate cancer from the group consisting of watchful waiting, radiotherapy with/without hormonal therapy, radical prostactomy with/without hormonal therapy and hormonal therapy;
b) carrying out treatment; and
c) performing on the patient ¹³C-MR detection of the prostate and optionally of surrounding organs and/or the surrounding area using an imaging medium that comprises hyperpolarized ¹³C-pyruvate, wherein signals ¹³C-pyruvate and its ¹³C-containing metabolites alanine, lactate and optionally bicarbonate are detected to monitor treatment response.

If step c) reveals that the treatment has not been fully successful, steps b) and c) may be repeated (follow-up treatment) until the desired results are achieved. The follow-up treatment may or may not be identical with the initial treatment.

In a preferred embodiment of the methods according to the eleventh aspect of the invention, the selected treatment is minimal invasive or non-invasive, more preferably cryosurgery, laser treatment or high intensity focused ultrasound (HIFU), most preferably MR guided high intensity focused ultrasound.

A more preferred embodiment of the present invention according to the eleventh aspect is a method of management of patients with a non-metastatic prostate tumor, said method comprises
a) acquiring MR images and/or MR spectra of the prostate and optionally of surrounding organs and/or the surrounding area;
b) performing thermal ablation treatment of the tumor using MR guided high intensity focused ultrasound; and
c) following the treatment, performing on the patient ¹³C-MR detection of the prostate and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to evaluate the treatment of step b); and
d) repeating step c) to monitor the treatment response.

In a thirteenth aspect, the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease;
c) if the stage of the disease has been determined to be metastatic in step b), selecting treatment from the group consisting of hormonal therapy and orchidectomy;
d) carrying out treatment; and
e) repeating step a) to monitor treatment response.

If step e) reveals that the treatment has not been fully successful, steps d) and e) may be repeated (follow-up treatment) until the desired results are achieved. The follow-up treatment may or may not be identical with the initial treatment.

A preferred embodiment of the present invention according to the thirteenth aspect is a method of management of patients with metastatic prostate cancer, said method comprises
a) selecting a treatment from the group consisting of hormonal therapy and orchidectomy;
b) carrying out treatment; and
c) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected to monitor treatment response.

If step c) reveals that the treatment has not been fully successful, steps b) and c) may be repeated (follow-up treatment) until the desired results are achieved. The follow-up treatment may or may not be identical with the initial treatment.

In a fourteenth aspect, the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease;
c) if the stage of the disease has been determined to be hormone refractory in step b) selecting treatment from the group consisting of chemotherapy and investigational therapy;
d) carrying out treatment; and
e) repeating step a) to monitor treatment response.

If step e) reveals that the treatment has not been fully successful, steps d) and e) may be repeated (follow-up treatment) until the desired results are achieved. The follow-up treatment may or may not be identical with the initial treatment.

A preferred embodiment of the present invention according to the fourteenth aspect is a method of management of patients with hormone refractory prostate cancer, said method comprises
a) selecting a treatment from the group consisting of chemotherapy and investigational therapy;
b) carrying out treatment; and
c) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected to monitor treatment response.

If step c) reveals that the treatment has not been fully successful, steps b) and c) may be repeated (follow-up treatment) until the desired results are achieved. The follow-up treatment may or may not be identical with the initial treatment.

Further, ¹³C-MR detection using a hyperpolarized MR imaging agent that comprises ¹³C-pyruvate of patients with cancer and having a tumor may be used for monitoring and detection of early recurrence of the cancer/tumor. In a preferred embodiment, said ¹³C-MR detection for monitoring and detection of early recurrence of cancer/of a tumor is carried out 12 month or more after finishing of the treatment. Preferably, ¹³C-MR detection using a hyperpolarized MR imaging agent that comprises ¹³C-pyruvate of patients with prostate cancer may be used for monitoring and detection of early recurrence of the prostate cancer. Risk factors for recurrence of prostate cancer and/or increased risk of metastatic disease are short time to biochemical recurrence, rapid PSA-DT, i.e. the time it takes for a PSA value to double after PSA returns to measurable levels following therapy of curative intent and high Gleason score. MR imaging and/or spectroscopy with an imaging agent that comprises ¹³C-pyruvate of patients with prostate cancer who had treatment may help to detect early recurrence, either alone or in combination with established methods like PSA determination.

Recurrence of cancer/the tumor manifests itself by a newly developed tumor in the same organ (e.g. a newly developed tumor in the prostate) or at the same area which can be seen on the MR images. Further cancer recurrence can be detected by the increased metabolic rate of tumor tissue, i.e. by an increase in the ¹³C-lactate signal. Recurrence may also be determined by comparing the ¹³C-lactate signal of the acquired MR images and/or MR spectra shortly after finishing the treatment and the MR images and/or MR spectra acquired for the purpose of checking on cancer recurrence. An increase in the ¹³C-lactate signal indicates an elevated metabolic rate and thus is an indicator for tumor tissue.

Thus, in a fifteenth aspect the present invention provides a method of management of patients with cancer having a tumor, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of cancer to determine the stage of disease;
c) selecting the treatment of the cancer;
d) carrying out treatment; and
e) repeating step a) to monitor recurrence of the cancer.

The method of the previous paragraph is preferably a method of management of patients with prostate cancer and said method thus comprises performing ¹³C-MR detection of the prostate in step a) and e).

If step e) reveals that the treatment has not been fully successful, steps d) and e) may be repeated (follow-up treatment) until the desired results are achieved. The follow-up treatment may or may not be identical with the initial treatment.

In another embodiment, the method according to the fifteenth aspect of the invention is a method of monitoring recurrence of cancer in patients having had a tumor, preferably of prostate cancer patients, and having had received treatment for said cancer, the method comprising performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected.

In a sixteenth aspect the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease; and
c) if the stage of the disease has been determined to be non-metastatic in step b), selecting a treatment of the prostate cancer from the group consisting of watchful waiting, radiotherapy with/without hormonal therapy, radical prostactomy with/without hormonal therapy and hormonal therapy;
d) carrying out treatment; and
e) repeating step a) to monitor recurrence of prostate cancer.

If step e) reveals that the treatment has not been fully successful, steps d) and e) may be repeated (follow-up treatment) until the desired results are achieved. The follow-up treatment may or may not be identical with the initial treatment.

A preferred embodiment of the method according to the sixteenth aspect the invention is a method of management of patients with non-metastatic prostate cancer, said method comprises
a) selecting a treatment of the prostate cancer from the group consisting of watchful waiting, radiotherapy with/without hormonal therapy, radical prostactomy with/without hormonal therapy and hormonal therapy;
b) carrying out treatment; and
c) performing on the patient ¹³C-MR detection of the prostate and optionally of surrounding organs and/or the surrounding area using an imaging medium that comprises hyperpolarized ¹³C-pyruvate, wherein signals of ¹³C-pyruvate and its ¹³C-containing metabolites alanine, lactate and optionally bicarbonate are detected to monitor recurrence of prostate cancer.

If step c) reveals that the treatment has not been fully successful, steps b) and c) may be repeated (follow-up treatment) until the desired results are achieved. The follow-up treatment may or may not be identical with the initial treatment.

In a preferred embodiment of the methods according to the sixteenth aspect of the invention, the selected treatment is an investigational approach, more preferably cryosurgery, laser treatment or high intensity focused ultrasound (HIFU), most preferably MR guided high intensity focused ultrasound.

A more preferred embodiment of the present invention according to the sixteenth aspect is a method of management of patients with non-metastatic prostate tumor, said method comprises
a) acquiring MR images and/or MR spectra of the prostate and optionally of surrounding organs and/or the surrounding area;
b) performing thermal ablation treatment of the tumor using MR guided high intensity focused ultrasound;
c) following the treatment, performing on the patient ¹³C-MR detection of the prostate and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to evaluate the treatment of step b); and
d) repeating step c) to monitor recurrence of prostate cancer.

If step c) reveals that the treatment has not been fully successful, steps b) and c) may be repeated (follow-up treatment) until the desired results are achieved. The follow-up treatment may or may not be identical with the initial treatment.

In a seventeenth, the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected
b) using information obtained in step a) optionally together with other information obtained in investigations carried out to confirm diagnosis of prostate cancer to determine the stage of disease;
c) if the stage of the disease has been determined to be metastatic in step b), selecting a treatment from the group consisting of hormonal therapy and orchidectomy;
d) carrying out treatment; and
e) repeating step a) to monitor recurrence of prostate cancer.

If step e) reveals that the treatment has not been fully successful, steps d) and e) may be repeated (follow-up treatment) until the desired results are achieved. The follow-up treatment may or may not be identical with the initial treatment.

A preferred embodiment of the present invention according to the seventeenth aspect is a method of management of patients with metastatic prostate cancer, said method comprises
a) selecting a treatment from the group consisting of hormonal therapy and orchidectomy;
b) carrying out treatment; and
c) performing on the patient ¹³C-MR detection of the prostate and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to monitor recurrence of prostate cancer.

If step c) reveals that the treatment has not been fully successful, steps b) and c) may be repeated (follow-up treatment) until the desired results are achieved. The follow-up treatment may or may not be identical with the initial treatment.

In an eighteenth aspect, the present invention provides a method of management of patients with prostate cancer, said method comprises
a) performing on the patient ¹³C-MR detection of the prostate and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of prostate cancer to determine the stage of disease;
c) if the stage of the disease has been determined to be hormone refractory in step b) selecting a treatment from the group consisting of chemotherapy and investigational therapy;
d) carrying out treatment; and
e) repeating step a) to monitor recurrence of prostate cancer.

If step e) reveals that the treatment has not been fully successful, steps d) and e) may be repeated (follow-up treatment) until the desired results are achieved. The follow-up treatment may or may not be identical with the initial treatment.

A preferred embodiment of the present invention according to the eighteenth aspect is a method of management of patients with hormone refractory prostate cancer, said method comprises
a) selecting a treatment from the group consisting of chemotherapy and investigational therapy;
b) carrying out treatment; and
c) performing on the patient ¹³C-MR detection of the prostate and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to monitor recurrence of prostate cancer.

If step c) reveals that the treatment has not been fully successful, steps b) and c) may be repeated (follow-up treatment) until the desired results are achieved. The follow-up treatment may or may not be identical with the initial treatment.

A preferred embodiment is a method of management of patients with cancer having a tumor, preferably of patients with prostate cancer, is a combined approach of the methods according to the invention.

Hence in a nineteenth aspect the present invention provides a method of management of patients with cancer having a tumor, said method comprises
a) performing on the patient ¹³C-MR detection of the tumor and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected;
b) using information obtained in step a) optionally together with other information obtained in investigations carried out to confirm diagnosis of cancer to determine the stage of disease;
c) selecting the treatment of the cancer;
d) carrying out treatment;
e) within a period of about 168 hours after step d) repeating step a) to monitor the early treatment response;
f) optionally repeating step d) and optionally step e) until the desired treatment result is achieved;
g) within a period of about 3 to 12 month after step d) or step f) repeating step a) to monitor treatment response;
h) optionally repeating step d) and optionally step e) and/or g) until the desired treatment result is achieved;
i) within a period of more than 12 month after step d) or step f) or step h) repeating step a) to monitor recurrence of cancer.

The method of the previous paragraph is preferably a method of management of patients with prostate cancer and said method thus comprises performing on the patient ¹³C-MR detection of the prostate in step a), e), g) and i).

The treatment in steps d), f) and h) may or may not be identical.

In another embodiment, the method according to the nineteenth aspect of the invention is a method of management of patients with cancer having a tumor and receiving or having received treatment for said cancer, the method comprising
a) within a period of about 168 hours after said treatment performing on the patient ¹³C-MR detection of the tumor and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to monitor the early treatment response;
b) optionally repeating the treatment or using a different treatment and optionally repeating step a) until the desired treatment result is achieved;
c) within a period of about 3 to 12 month after the treatment or step b) performing on the patient ¹³C-MR detection of the tumor and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to monitor treatment response;
d) optionally repeating the treatment or using a different treatment and optionally repeating step a) and/or step c) until the desired treatment result is achieved;
e) within a period of more than 12 month after the treatment or step b) or step d) performing on the patient ¹³C-MR detection of the tumor and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to monitor recurrence of cancer.

The method of the previous paragraph is preferably a method of management of patients with prostate cancer and said method thus comprises performing on the patient ¹³C-MR detection of the prostate in step a), c) and e).

Yet another aspect of the invention are medical algorithms for patients with cancer having a tumor, preferably for patients with prostate cancer, corresponding to the methods of the invention. Said medical algorithms include decision trees, flow diagrams or are computerized algorithms which help to clarify the different options for each stage of diagnosis and optionally treatment, disease staging and management of cancer according to the methods of the invention.

The medical algorithms according to the invention can be represented on paper but also on a computer readable storage medium.

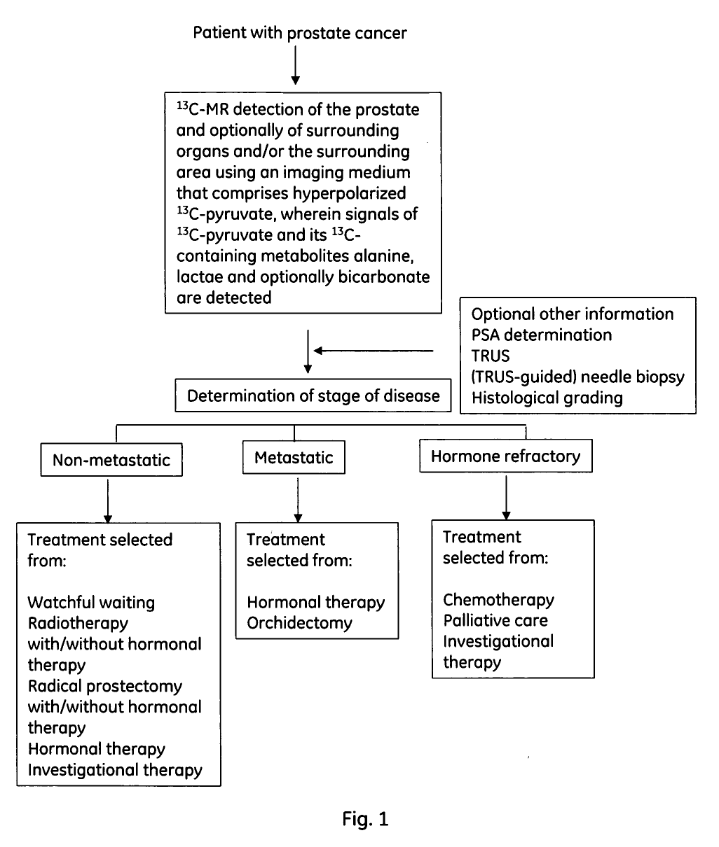

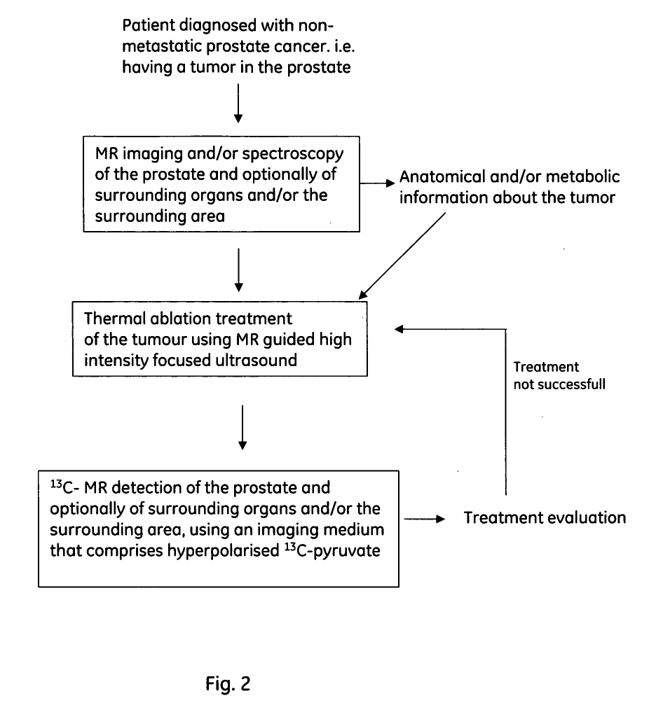

Two examples of such an algorithm are illustrated in FIG. 1 and FIG. 2.

FIG. 1 shows an algorithm for patients with prostate cancer corresponding to the preferred embodiment of the method of the second aspect of the invention.

FIG. 2 shows an algorithm for patients with prostate cancer corresponding to the preferred embodiment of the method of the sixth aspect of the invention.

What is claimed is:

1. Method of management of patients with cancer having a tumor, said method comprises
a) performing on the patient ¹³C-MR detection of the tumor and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected and;
b) using information obtained in step a) optionally together with other information obtained in investigations carried out to confirm diagnosis of cancer to determine the stage of disease.

2. Method according to claim 1 comprising as a further step c) selecting the treatment of the cancer.

3. Method according to claim 2 comprising the further steps d) carrying out treatment; and e) repeating step a) to monitor the early treatment response, and/or treatment response and/or the recurrence of the cancer.

4. Method according to claim 1 wherein said patient is a patient with prostate cancer and ¹³C-MR detection in step a) is a ¹³C-MR detection of the prostate.

5. Method of management of patients with non-metastatic cancer having a tumor, said method comprises
a) acquiring MR images and/or MR spectra of the tumor and optionally of surrounding organs and/or the surrounding area;
b) performing thermal ablation treatment of the tumor using MR guided high intensity focused ultrasound; and
c) following the treatment, performing on the patient ¹³C-MR detection of the tumor and optionally of surrounding organs and/or the surrounding area using an imaging medium that comprises hyperpolarized ¹³C-pyruvate wherein signals of ¹³C-pyruvate and its ¹³C-containing metabolites alanine, lactate and optionally bicarbonate are detected to evaluate the treatment of step b).

6. Method according to claim 5 comprising a further step d) repeating step c) to monitor the early treatment response, and/or treatment response and/or the recurrence of the cancer.

7. Array of devices in a MR scanner room comprising a MR scanner, a patient table, a DNP polarizer, an injector and a transducer that generates focused ultrasound beams.

8. Array according to claim 7 wherein the patient table is docked to the MR scanner, the DNP polarizer is for producing a MR imaging medium comprising a hyperpolarized imaging agent, preferably comprising hyperpolarized ¹³C-pyruvate, and the injector is connected to said DNP polarizer for injecting the MR imaging medium into a patient on said patient table.

9. Array according to claim 7 wherein said transducer is incorporated into said patient table and said patient table further comprises an automated positioning system.

10. Medical algorithms for patients with cancer having a tumor, preferably for patients with prostate cancer, corresponding to the methods of the invention.

11. Medical algorithms according to claim 10 wherein said medical algorithms are on a computer readable storage medium.
